(19) United States
(12) Patent Application Publication
(10) Pub. No.: US 2011/0124719 A1
(43) Pub. Date: May 26, 2011

(54) COMPOSITION FOR PROMOTING PRODUCTION OF HYALURONIC ACID CONTAINING KAEMPFEROL AND QUERCETIN

(75) Inventors: Seung-Hun KIM, Seoul (KR); Byung-Young KANG, Seoul (KR); Gae-Won NAM, Yongin-city (KR); Hae-Kwang LEE, Woowon-City (KR); Seong-Joon MOON, Goonpo-city (KR); Ih-Seop JANG, Yongin-city (KR)

(73) Assignee: Amorepacific Corporation, Seoul (KR)

(21) Appl. No.: 12/946,417

(22) Filed: Nov. 15, 2010

Related U.S. Application Data
(62) Division of application No. 11/813,186, filed on May 21, 2008, now abandoned.

(30) Foreign Application Priority Data
Dec. 31, 2004 (KR) 10-2004-0117888

(51) Int. Cl. A61K 31/352; A61K 8/49; A61P 19/02; A61Q 19/08; A61Q 19/00

(52) U.S. Cl. 514/456

(57) ABSTRACT

Disclosed is a composition for promoting a production of hyaluronic acid containing at least one of kaempferol and quercetin. Kaempferol and quercetin of the invention increase an expression of a hyaluronic acid synthase (HAS) gene existing in a skin cell line of human epidermis, thereby promoting a production of hyaluronic acid in the human cell. Accordingly, the composition containing at least one of kaempferol and quercetin according to the invention can be usefully used as a cosmetic composition for increasing skin elasticity and preventing skin dryness or aging, or a pharmaceutical composition for treating or preventing a degenerative arthritis.

### CROSS-REFERENCE TO RELATED APPLICATIONS

This application is a Divisional Application of U.S. Ser. No. 11/813,186, filed May 21, 2008, which is a National Stage Application of PCT/KR2005/01592, filed May 30, 2005, which claims benefit of Ser. No. 10-2004-0117888, filed Dec. 31, 2004 in the Republic of Korea and which applications are incorporated herein by reference. A claim of priority to all, to the extent appropriate is made.

### TECHNICAL FIELD

The present invention relates to a composition for promoting a production of hyaluronic acid containing at least one of kaempferol and quercetin.

### BACKGROUND ART

Hyaluronic acid is a kind of nonsulfated glycosaminoglycans having no sulfuric acid groups bonded thereto, and a linear polymer material having a molecular weight of 200,000-400,000 where glucuronic acid and N-acetylglucosamine residues are repeatedly connected in the form of chain. It was reported that hyaluronic acid was a main ingredient of an extracellular matrix and involved in division, differentiation and movement of a cell as well as moisture retention, maintenance of a space between cells, storage and spread of a cell growth factor and nutritive substances.

It was reported that 50% or more of hyaluronic acids existing in the body of a mammal were distributed a skin, particularly, intercellular space of epidermis and a connective tissue of dermis. It was also known that hyaluronic acid was synthesized mainly by keratinocyte and fibroblast. It was reported that an amount of the hyaluronic acid was reduced according to the aging in the human skin. It is believed that the reduction of the amount of hyaluronic acid is one of direct causes of loss of skin elasticity and decrease of moisture content according to the aging (Fleischmajer R. et al., Biochem Biophys Acta., 279, pp 265-275, 1972; Longas M O. et al., Carbohydr Res., 159, pp 127-136, 1987; Ghersetich I. et al., Int. J. Dermatol., 33, pp 119-122, 1994).

A joint capsule of the human body consists of an outer fibrous layer and an inner synovial layer, wherein synovial fluid produced in the synovial layer contains hyaluronic acid (hyaluronate) and glycoprotein which serve to lubricate a joint. When there occurs a degenerative arthritis, the generation of the hyaluronic acid serving as a lubricant in the joint is decreased and destructions by a proteolytic enzyme are increased to decrease the hyaluronic acid in the joint. In other words, as the hyaluronic acid in the joint is decreased, the joint cannot absorb or disperse a shock from an exterior, so that a damage of the joint may be more severe. Regarding this, a method of injecting hyaluronic acid into a joint to alleviate the arthritis was approved by U.S. FDA in 1997 and have been currently performed. However, it may be more effective to increase a synthesis of the hyaluronic acid in the human body.

It was reported that a synthesis of the hyaluronic acid under state of skin cell culture was increased by various kinds of growth factors, transretinoic acid and N-methylserine, etc. (Heldin P. et al., Biochem, J., 258, pp 919-922, 1989; Heldin P. et al., Biochem, J., 283, pp 165-170, 1992; Suzuki M. et al., Biochem, J., 307, pp 817-821, 1995; Tirone E. et al., J. Biol. Chem., 272, pp 4787-4794, 1997; Tammi R. et al., J. Invest. Dermatol., 92, pp 326-332, 1989; Akiyama H. et al., Biol. Pharm. Bull., 17, pp 361-364, 1994; Sakai S. et al., Skin Pharmacol. Appl. Skin Physiol., 12, pp 276-283, 1999). It was also reported that estradiol and similar substances applied to the skin increased the synthesis of the hyaluronic acid (Sobel H. et al., Steroids, 16, pp 1-3, 1970; Bentley J P. et al., J. Invest. Dermatol., 87, pp 668-673, 1986; Miyazaki K. et al., Skin Pharmacol. Appl. Skin Physiol., 15, pp 175-183, 2002). However, it has not yet been known of a detailed mechanism of the metabolism of the hyaluronic acid. It was just known that the synthesis of the hyaluronic acid is progressed by hyaluronic acid synthase in an inner surface of a cell membrane, and the hyaluronic acid goes out of the cell membrane in the progress of synthesis and accumulates in the extracellular matrix (Weigel P H. et al., J. Biol. Chem., 272, pp 13997-14000, 1997).

As genes of the hyaluronic acid synthase in a mammal, it was reported three types of hyaluronan synthase 1 (HAS1), hyaluronan synthase 2 (HAS2) and hyaluronan synthase 3 (HAS3) having a high sequence similarity. In this regard, it was reported that when an epidermal growth factor (EGF) was added to a culture solution of epidermal cells, a gene expression of the hyaluronic acid synthase (HAS) was increased (Pienimaki J P. et al. J. Biol. Chem., 276, pp 20428-20435, 2001). However, it has been quite insufficient to research distributions of hyaluronic acid in a cell and a tissue, and various factors and enzymes relating to hyaluronic acid, for example, the hyaluronic acid synthase (HAS) or factors regulating an activity of hyaluronic acid.

Considering the applicabilities of hyaluronic acid, although it was actively performed researches on a method for effectively preparing and injecting the hyaluronic acid or increasing a synthesis of the hyaluronic acid in a human body, a remarkable research result has not been reported yet.

Kaempferol having a following chemical formula 1 and quercetin having a following chemical formula 2, which are kinds of flavonols which are flavonoids, are general edible polyphenol compounds, exist much in edible plants and are known to play an important role in health. In general, flavonoids having the diphenylpropane skeleton are also known to have anti-cancer, anti-oxidization, anti-inflammatory and anti-allergic efficacies.

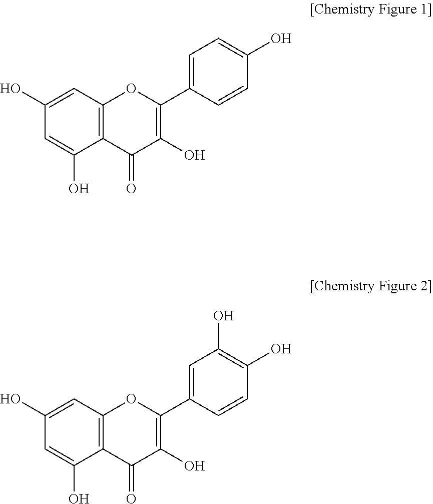

### DISCLOSURE

### Technical Problem

The inventors have steadily researched on a method for providing hyaluronic acid to a human body more effectively. As a result of that, the inventors found that kaempferol and quercetin, which are ones of flavonoids and known to have anti-cancer, anti-oxidization, anti-inflammatory and anti-allergic efficacies, had an efficacy of increasing an expression of a gene encoding hyaluronic acid synthase in a cell of the human body and thus promoting a production of hyaluronic acid in the human body as well as the known efficacies.

In other words, it was found that since production of hyaluronic acid by cells was promoted and an amount of hyaluronic acid in the human body was increased when a skin cell line of human epidermis was treated with kaempferol or quercetin, the hyaluronic acid could be used for various uses, for example a drug for treating or preventing a degenerative arthritis or for improving skin such as skin elasticity improvement and prevention of skin dryness or aging.

Accordingly, the object of the present invention is to provide a composition for promoting a production of hyaluronic acid containing kaempferol and quercetin as effective ingredients.

Another object of the invention is to provide various uses using an efficacy of hyaluronic acid synthase of the composition for promoting a production of hyaluronic acid, for example, availabilities of kaempferol and quercetin for treatment or prevention of a degenerative arthritis or for skin improvement such as skin elasticity improvement and prevention of skin dryness or aging.

### Technical Solution

In order to accomplish the object, there is provided a composition for promoting a production of hyaluronic acid containing at least one of kaempferol and quercetin.

The composition for promoting a production of hyaluronic acid increases an expression of a hyaluronic acid synthase (HAS) gene, thereby promoting the production of hyaluronic acid.

In addition, according to an embodiment of the invention, the composition for promoting a production of hyaluronic acid may be a cosmetic composition for improving skin elasticity.

Further, according to an embodiment of the invention, the composition for promoting a production of hyaluronic acid may be a cosmetic composition for preventing skin dryness.

Additionally, according to an embodiment of the invention, the composition for promoting a production of hyaluronic acid may be a cosmetic composition for preventing skin aging.

In addition, according to an embodiment of the invention, the composition for promoting a production of hyaluronic acid may be a pharmaceutical composition for treating or preventing a degenerative arthritis.

Preferably, the composition for promoting a production of hyaluronic acid may contain at least one of kaempferol and quercetin in a concentration of 0.001-99.9 wt. %, based on a total weight of the composition. When the concentration is less than 0.001 wt. %, it is difficult to obtain an efficacy thereof, and when the concentration is more than 99.9 wt. %, there may occur a problem of formulation stability and the concentration cannot excess 99.9 wt. % due to the presence of impurities.

### Advantageous Effects

Kaempferol and quercetin, which are flavonoids, increase an expression of a HAS gene existing in a skin cell line of human epidermis, thereby promoting a production of hyaluronic acid. Accordingly, the composition for promoting production of hyaluronic acid containing at least one of kaempferol and quercetin according to the invention can be usefully used as a cosmetic composition for increasing skin elasticity and preventing skin dryness or skin aging, or a pharmaceutical composition for treating or preventing a degenerative arthritis.

### DESCRIPTION OF DRAWINGS

FIGS. 1 and 2 are results of quantitative reverse transcription PCR of hyaluronic acid synthase (HAS) genes after a skin cell line of human epidermis i.e., a keratinocyte cell line of HaCaT cells was treated with each of kaempferol and quercetin in several concentrations, in order to examine an expression of hyaluronic acid synthase by kaempferol and quercetin in a level of mRNA wherein FIG. 1 is a resultant view by kaempferol and FIG. 2 is a resultant view by quercetin;

FIG. 3 shows quantitatively an examination result of an increase of a production of hyaluronic acid using an ELISA (Enzyme-Linked ImmunoSorbent Assay) after a skin cell line of human epidermis, i.e., a keratinocyte cell line of HaCaT cells was treated with kaempferol and quercetin in concentrations of 0.1 μM, 1 μM and 10 μM, respectively, in order to examine production promotions of hyaluronic acid by kaempferol and querccetin; and

FIG. 4 shows quantitatively an examination result of influences of kaempferol and quercetin on cytotoxicity through a MTT{3,(4,5-dimethylthiazol-2-yl) 2,5-diphenyltetrazolium bromide} assay after a skin cell line of human epidermis, i.e., a keratinocyte cell line of HaCaT cells was treated with kaempferol and quercetin in concentrations of 0.1 μM, 1 μM and 10 μM, respectively, in order to examine cytotoxicity of kaempferol and quercetin.

### BEST MODE

According to the invention, in order to examine an effect of promoting a production of hyaluronic acid by kaempferol and quercetin which are flavonoids, a skin cell line of human epidermis, i.e., a keratinocyte cell line of HaCaT cells was treated with kaempferol and quercetin, respectively. As a result of that, it was seen that an expression of a hyaluronic acid synthase (HAS) gene was increased and the production of hyaluronic acid was also increased. In other words, it was confirmed that HaCaT cells, which were a skin cell line of human epidermis treated with kaempferol and quercetin for 24 hours, exhibited an increased expression of a hyaluronic acid synthase gene, compared to the cells which were not treated with them. This means that kaempferol and quercetin have an efficacy of promoting an expression of a hyaluronic acid synthase gene in the skin cells of human epidermis. At the same time, it was also confirmed that an amount of hyaluronic acid was increased due to the treatment of kaempferol and quercetin in the skin cell line of human epidermis.

Accordingly, kaempferol and quercetin having efficacies of increasing an expression of a hyaluronic acid synthase gene and promoting a production of hyaluronic acid can be used as an effective ingredient of various skin external preparations using a usability of hyaluronica acid. For example, they may be added to a cosmetic composition for increasing skin elasticity and preventing skin dryness or aging.

In addition, kaempferol and quercetin may be added to a pharmaceutical composition for treating or preventing a disease such as a degenerative arthritis with the administration of hyaluronic acid. However, it should be noted that the present invention is not limited to this.

A cosmetic composition containing at least one of kaempferol and quercetin according to the invention may preferably comprise other ingredients capable of providing a synergy effect to the main effect of the invention within a range of not exerting a bad influence on the main effect, in addition to the above ingredients.

Additionally, the cosmetic composition may have a form such as a solution, an emulsion and a viscous mixture, etc.

The cosmetic composition of the invention may include a skin adhesive type cosmetic such as emulsion, skin water, cream, lotion, essence, pack and gel, a cosmetic having a formulation such as powder, lip stick, makeup base and foundation and a cleaning cosmetic such as shampoo, rinse, body cleanser, cosmetic solution, cleansing foam, cleansing cream, cleansing water and soap, but is not limited to them.

In the cosmetic composition of each formulations, other ingredients than at least one of kaempferol and quercetin may be optimally formulated without a difficulty by a skilled in the art according to formulations or the objects of uses of the cosmetics.

In addition, the cosmetic composition of the invention may comprise a composition selected from a group consisting of water soluble vitamin, fat soluble vitamin, polymer peptide, polymer polysaccharide, sphingolipid and algae extracts.

The cosmetic composition of the invention may be formulated with other ingredients, which are formulated to a typical cosmetic, as necessary together with the essential ingredients.

Other formulation ingredients, which can be added, may include fat and oil ingredients, moisturizer, emollient agent, surfactant, inorganic and organic pigments, organic powders, ultraviolet absorbing agent, antiseptic, sterilizer, anti-oxidant, plant extracts, pH adjustor, alcohol, pigment, flavor, blood circulation-promoting agent, cold sensation agent, anhydrotics and purified water.

Further, other formulation ingredients, which can be added, are not limited to the above ingredients and any ingredients as mentioned above can be formulated within a range of not exerting a bad influence on the objects and effects of the invention, but are preferably added in a range of 0.01˜5 wt. %, more preferably 0.01˜3 wt. % of the total weight.

A pharmaceutical composition containing at lease one of kaempferol and quercetin of the invention may further comprise a proper carrier, an excipient and a diluent typically used for preparing the pharmaceutical composition.

A pharmaceutical administration type of at least one of kaempferol and quercetin of the invention is as follows. That is, it may be used as a pharmaceutically acceptable salt thereof. Further, it may be used alone or together with other pharmaceutical active compounds in a form of a combination or proper set thereof.

A pharmaceutical composition containing at least one of kaempferol and quercetin according to the invention may be formulated into a type suitable for a pharmaceutical preparation, including an oral administration-type formulation such as powder, granulum, tablet, capsule, suspension, emulsion, syrup and aerosol, and a transdermal administration-type formulation such as lotion, ointment, gel, cream, patch and aerosol.

Although a preferable dosage of the pharmaceutical composition according to the invention is different according to ages, sexes, weights, symptoms and degrees of diseases, drug forms, administration routes and administration periods, it can be properly selected by a skilled in the art. However, considering a preferable effect, it is preferred that the pharmaceutical composition of the invention is administrated in an amount of 0.01˜1000 mg/kg per a day. The administration can be performed one time or many times per a day. In addition, the dosage can be increased or decreased according to the ages, sexes, weights, degrees of diseases and administration routes, etc. Accordingly, the dosage does not limit a scope of the invention in any way.

At least one of kaempferol and quercetin of the invention can be administrated to a mammal such as a rat, a mouse, a domestic animal and a human through various routes, for example, non-oral and oral administrations. All types of the administration can be expected. For instance, it can be administrated with oral, rectum or vein, muscle, hypodermic, and intrauterine dura mater or intracerebroventricular injections.

### MODE FOR INVENTION

Hereinafter, the invention will be more specifically described with experimental examples. However, it should be noted that the invention is not limited to the experimental examples.

### Experimental Example 1

### An Effect of Increasing an Expression of a Hyaluronic Acid Synthase (HAS) Gene in HaCaT which is a Skin Cell Line of Human Epidermis

In order to examine an effect of increasing an expression of a HAS gene by kaempferol and quercetin in HaCaT which is a skin cell line of human epidermis, HaCaT cells were respectively treated with kaempferol and quercetin in several concentrations, then the HAS gene was subject to quantitative reverse transcription PCR so as to examine an expression of the HAS gene in a level of mRNA and thus changes of the HAS genes by kaempferol and quercetin were examined as follows.

1-1. Cell Culture

Human keratinocyte cell line, HaCaT cell, which was spontaneously immortalized, was used and obtained from Dr. N. E. Fusenig (Deutsches Krebsforschungszentrum (DKFZ), Heidelberg, Germany).

Firstly, the cells were cultured in a Dulbecco's modified Eagle's media (DMEM) containing 10% fetal bovine serum (HyClone), sodium bicarbonate 3.6 g/l and antibody (streptomycin 100 μg and penicillin 100 IU/l) (Life Technologies, Inc.) under proper culture conditions (37° C., 5% CO₂, 95% air). The medium was replaced every three days and the cells were secondary-cultured in a division ratio of 1:5 as soon as the density thereof reached the highest. 1×10⁵ cells per 75 cm² of a tissue culture flask were aliquot 72 hours before the cell were treated with kaempferol or quercetin, and then cultured in a medium containing 10% fetal bovine serum for 48 hours. Then, the cells were cultured in a serum-free medium for 24 hours, treated with kaempferol and quercetin in concentrations of 0.1 M, 1 μM and 10 μM, respectively and then cultured for 24 hours. A control group was diluted in a ratio of 1:1,000 and cultured in a medium supplemented with vehicle (dimethylsulfoxide, DMSO). It was not observed any effects of DMSO influencing on growth and differentiation in the control group culture.

1-2. RNA Preparation

The HaCaT cells cultured in the experimental example 1-1 were cleaned two times with a phosphate-buffered saline (Life Technologies, Inc.) and RNAs of all cells were separated using a trizol reagent (GibcoBRL Life Technologies, Grand Island, N.Y.) according to manufacturer's instructions. A concentration of RNA was measured with a spectrophotometry method and a quality of RNA was checked with an agarose gel electrophoresis.

1-3. Measurement of Effects on mRNA Synthesis of Hyaluronic Acid Synthase Through a PCR

It was examined through a quantitative reverse transcription PCR kaempferol and quercetin's influences on mRNA synthesis of hyaluronan synthase 1 (NAS1), hyaluronan synthase 2 (HAS2) and hyaluronan synthase 3 (HAS3), which are isoforms of hyaluronic acid synthase, as follows.

Firstly, a RNA prepared and quantified in the above experimental example 1-2 was subject to a reverse transcription and then it was performed a quantitative PCR under the presence of primers specific for HAS1, HAS2 and HAS3 which are isoforms of hyaluronic acid synthase. More specifically, 4 μg of RNA was subject to a reverse transcription in 2 μl of reaction mixture containing 1 μl of M-MuLV reverse transcription polymerase (20 U/μl, MBI Fermentas), 1 μl of RNase inhibitor (20 U/μl), 4 μl of 5× reaction buffer, 2 μl of 10 mM dNTP mix and 1 μl of oligo (dT) primer (0.5 μg/μl) (it was performed according to manufacturer's instructions using a first strand cDNA synthesis kit #1612 of MBI Fermentas). The initial RNA, oligo (dT) primer and DEPC-H₂O were mixed to be 11 μl, reacted at 70° C. for 5 minutes and then put into an ice. After that, 5× reaction buffer, RNase inhibitor and dNTP were put and reacted at 37° C. for 5 minutes, and then M-MuLV was put and again reacted at 37° C. for 60 minutes, so that the mixture was subject to a reverse transcription. After that, a heat treatment was performed at 70° C. for 10 minutes to eliminate the activity of a reverse transcriptase. Subsequently, it was taken 3 μl of the reactive mixture to use it in a PCR reaction. Each of PCRs was performed using a Perkin-Elmer Cycler 9600 (Perkin-Elmer Applied Biosystems, Foster, Calif.) in 20 μl of reaction mixture containing TaKaRa Ex Taq DNA polymerase (5 U/μl, TaKaRa), 10× Ex Taq Buffer, MgCl₂, dNTP mixture and 25 pM of a proper sense or antisense PCR primer (refer to Table 1).

TABLE 1

| Name of primer | | Sequence |
|---|---|---|
| HAS1 | Forward | 5-AGG TCA TGT ACA CAG CCT TC-3 |
| | Reverse | 5-CAG CAG AGG GAC GTA GTT AG-3 |
| HAS2 | Forward | 5-GCT ACC AGT TTA TCC AAA CG-3 |
| | Reverse | 5-GGA GTT TCT GTA CAT TCC CA-3 |
| HAS3 | Forward | 5-GAG GAC TGG TAC CAT CAG AA-3 |
| | Reverse | 5-ACC GTT CTT TGC ATT TTA GA-3 |

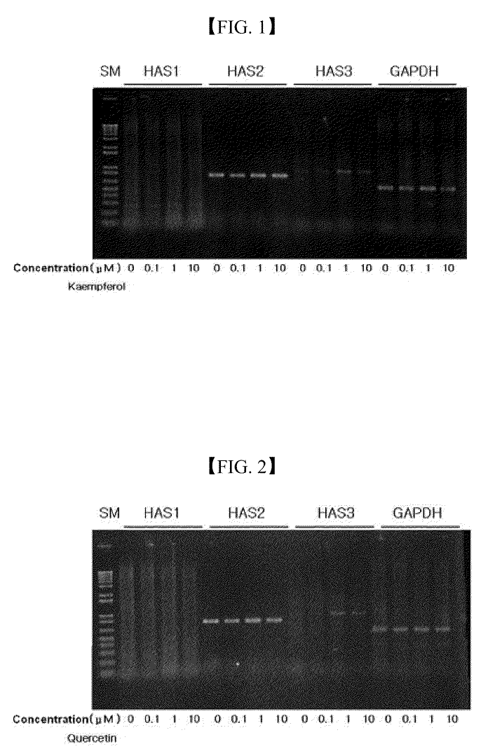

Reaction conditions of PCR were as follows. One denaturation cycle was performed at 94° C. for 5 minutes and then cycles were repeated 30 times at 94° C. for 1 minute, 55° C. for 1 minute and 72° C. for 1 minute and 30 seconds. A PCR result was subject to an agarose gel electrophoresis and then dyed with ethidium bromide. The results are shown in FIGS. 1 and 2. At this time, a result of GAPDH amplification was based for standardization.

As shown in FIGS. 1 and 2, it could be seen that HAS2 among the hyaluronic acid synthase genes was expressed in both kaempferol and quercetin treatment groups as well as the control group. Although HAS3 was detected a little in the control group, it was increased in HaCaT cells treated with 1 μM and 10 μM of kaempferol or quercetin, compared to the control group.

### Experimental Example 2

### An Increase of Hyaluronic Acid in Culture Medium of HaCaT Cell which is a Skin Cell Line of Human Epidermis

In order to examine that a production of hyaluronic acid was actually promoted by kaempferol and quercetin, a culture medium of HaCaT cell, which is a skin cell line of human epidermis was treated with kaempferol and quercetin, in several concentrations. After that, an amount of hyaluronic acid that synthesis of hyaluronic acid is promoted and then the acid is discharged into the medium was quantified using HA-ELISA Kit.

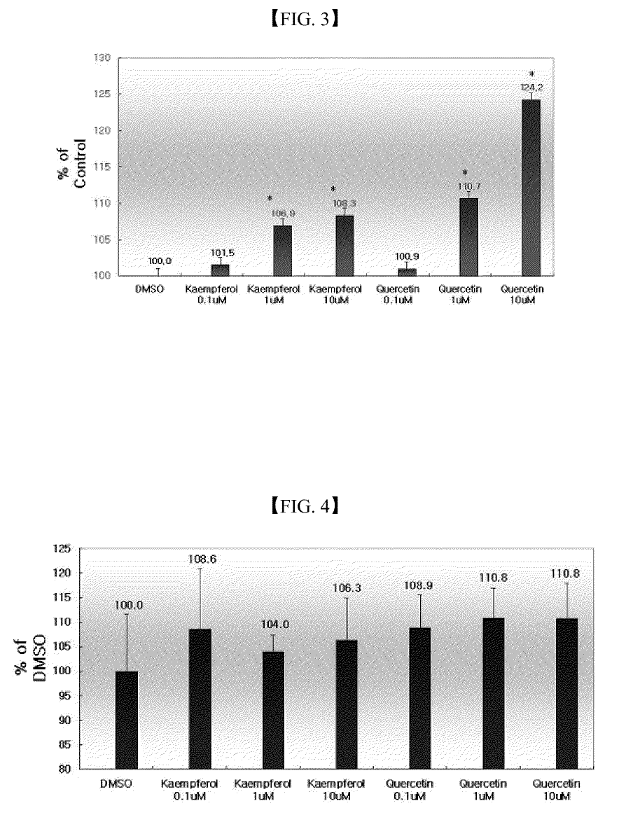

More specifically, HaCaT which is a skin cell line of human epidermis was subject to a cultivation according to the cell culture method of the experimental example 1-1 and then the culture medium was recovered to perform an ELISA (Enzyme-Linked ImmunoSorbent Assay), in order to examine an amount of hyaluronic acid that synthesis of hyaluronic acid was promoted by kaempferol and quercetin and thus the acid was discharged into the medium. To perform the ELISA, it was purchased a Hyaluronan Enzyme-Linked ImmunoSorbent Assay Kit (HA-ELISA, Product No.: K-1200) available from Echelon company. The medium recovered through the above cell cultivation was subject to the HA-ELISA Kit according to manufacturer's instructions, thereby quantifying the synthesis amount of hyaluronic acid that was synthesized in HaCaT cell by kaempferol and quercetin and then discharged into the medium. The control group was diluted in a ratio of 1:1,000 and then cultured in a medium supplemented with vehicle (dimethylsulfoxide, DMSO). It was not observed any effects of DMSO influencing on growth and differentiation in the control group culture. The result is shown in FIG. 3.

As shown in FIG. 3, it could be seen that synthesis ability of kaempferol was increased by 7% and 8%, respectively, at 1 μM and 10 compared to the control group, which is statistically significant with a reliability of 95%, and that synthesis ability of quercetin was increased by 11% and 24%, respectively, at 1 μM and 10 μM, compared to the control group, which is statistically significant with a reliability of 95%.

### Experimental Example 3

### An Examination of Cytotoxicity of Kaempferol and Quercetin in HaCaT, which is a Skin Cell Line of Human Epidermis, According to the Concentrations thereof

In order to examine cytotoxicity of kaempferol and quercetin in HaCaT, which is a skin cell line of human epidermis, according to the concentrations thereof, HaCaT was treated with kaempferol and quercetin in several concentrations and then degrees of cytotoxicity were quantified through a MTT assay.

3-1. Cell Culture

HaCaT of human keratinocyte cell line, which was spontaneously immortalized, was used and obtained from Dr. N. E. Fusenig (Deutsches Krebsforschungszentrum (DKFZ), Heidelberg, Germany). The cells were cultured in a Dulbecco's modified Eagle's media (DMEM) containing 10% fetal bovine serum (HyClone), sodium bicarbonate 3.6 g/l and antibody (streptomycin 100 μg and penicillin 100 IU/l) (Life Technologies, Inc.) under proper culture conditions (37° C., 5% CO₂, 95% air). The medium was replaced every three days and the cells were secondary-cultured in a division ratio of 1:5 as soon as the density thereof reached the highest. 1×10⁴ cells per well of a tissue culture flask 96 well plate were aliquot 72 hours before the cells were treated with kaempferol or quercetin, and then cultured in a medium containing 10% fetal bovine serum for 48 hours. Then, the cells were cultured for 24 hours in a serum-free medium, treated with kaempferol and quercetin in concentrations of 0.1 μM, 1 μM and 10 μM, respectively and then cultured for 24 hours. A control group was diluted in a ratio of 1:1,000 and cultured in a medium supplemented with vehicle (dimethylsulfoxide, DMSO).

3-2. MTT Assay

The cells cultured in the experimental example 3-1 was cleaned with phosphate-buffered saline and then subject to MTT {3,(4,5-dimethylthiazol-2-yl) 2, 5-diphenyltetrazolium bromide, Sigma} assay according to manufacturer's instructions. The result is shown in FIG. 4.

As shown in FIG. 4, it was not observed any effects of DMSO influencing on growth and differentiation in the control group culture. It could be also seen that kaempferol and quercetin did not exhibit cytotoxicity at 0.1 μM, 1 μM and 10 μM, which are synthesis evaluation concentrations of hyaluronic acid, compared to the control group, which is statistically significant with a reliability of 95%.

### Reference Example 1

### Statistical Analysis

A paired t-test, which was used in the data analysis in the experimental examples 1 to 3, was performed using a SigmaStat (SPSS, Inc., Chicago, Ill.). A significance was considered, based on p=0.05 and data was shown with mean±standard error.

Through the above result, it could be seen that kaempferol and quercetin increased the expression of HAS genes when a skin cell line of human epidermis was treated with them and thus promoted a production of hyaluronic acid.

Hereinafter, preparations examples of the above composition will be explained. However, it should be noted that the examples are provided to specifically illustrate the invention, not to limit the invention.

### Preparation Example 1

### Soap Preparation

| | |
|---|---|
| Kaempferol | 1.00% |
| Fat and oil | proper quantity |
| Sodium hydroxide | proper quantity |
| Sodium chloride | proper quantity |
| Flavor | small amount |

A total amount was 100 by the addition of purified water and soap was prepared according to the above formulation ratio.

### Preparation Example 2

### Lotion Preparation

| | |
|---|---|
| Quercetin | 3.00 (%) |
| L-ascorbic acid-2-magnesium phosphate salt | 1.00 |
| Water soluble collagen (1% aqueous solution) | 1.00 |
| Sodium citrate | 0.10 |
| Citric acid | 0.05 |
| Licorice extract | 0.20 |
| 1,3-butyleneglycol | 3.00 |

A total amount was 100 by the addition of purified water and lotion was prepared according to the above formulation ratio (%).

### Preparation Example 3

### Cream Preparation

| | |
|---|---|
| Kaempferol and quercetin | 1.00 (%) |
| Polyethylene glycol mono stearate | 2.00 |
| Self-emulsifying mono stearate glycerin | 5.00 |
| Cetyl alcohol | 4.00 |
| Squalene | 6.00 |
| tri2-ethyl hexane glyceryl | 6.00 |
| Sphingoglycolipid | 1.00 |
| 1,3-butyleneglycol | 7.00 |

A total amount was 100 by the addition of purified water and cream was prepared according to the above formulation ratio (%).

### Preparation Example 4

### Pack Preparation

| | |
|---|---|
| Kaempferol | 2.00 (%) |
| Polyvinyl alcohol | 13.00 |
| L-ascorbic acid-2-magnesium phosphate salt | 1.00 |
| Lauroylhydroxyproline | 1.00 |
| Water soluble collagen (1% aqueous solution) | 2.00 |
| 1,3-butyleneglycol | 3.00 |
| Ethanol | 5.00 |

A total amount was 100 by the addition of purified water and pack was prepared according to the above formulation ratio (%).

### Preparation Example 5

### Cosmetic Solution Preparation

| | |
|---|---|
| Quercetin | 2.00 (%) |
| Hydroxyethylene cellulose | 12.00 |
| Xanthan gum (2% aqueous solution) | 2.00 |
| 1,3-butyleneglycol | 6.00 |
| concentrated glycerin | 4.00 |
| Sodium hyaluronic acid (1% aqueous solution) | 1.00 |

A total amount was 100 by the addition of purified water and cosmetic solution was prepared according to the above formulation ratio (%).

### Preparation Example 6

### Powders Preparation

| | |
|---|---|
| Kaempferol | 100 mg |
| Lactose | 100 mg |
| Talc | 10 mg |

The above ingredients were mixed and filled in an air-tight pack to prepare powders.

### Preparation Example 7

### Tablet Preparation

| | | |
|---|---|---|
| Quercetin | 50 | mg |
| Corn starch | 100 | mg |
| Lactose | 100 | mg |
| Magnesium stearate | 2 | mg |

The above ingredients were mixed to prepare a tablet by tabletting according to a typical tablet preparing method.

### Preparation Example 8

### Capsule Preparation

| | | |
|---|---|---|
| Kaempferol and quercetin | 50 | mg |
| Corn starch | 100 | mg |
| Lactose | 100 | mg |
| Magnesium stearate | 2 | mg |

The above ingredients were mixed and filled in a gelatin capsule to prepare a capsule according to a typical capsule preparing method.

### Preparation Example 9

### Injection Preparation

| | |
|---|---|
| Kaempferol and quercetin | 50 mg |
| Sterilized distilled water for injection | proper quantity |
| pH adjuster | proper quantity |

An injection was prepared with the above contents per a one ample (2 Ml) according to a typical injection preparing method.

### Preparation Example 10

### Liquid Drug Preparation

| | | |
|---|---|---|
| Quercetin | 100 | mg |
| Isomerized sugar | 10 | g |
| Mannitol | 5 | g |

| | |
|---|---|
| Purified water | proper quantity |

According to a typical liquid drug preparing method, the above ingredients were dissolved in purified water to be dissolved and a proper quantity of lemon perfume was added to the mixture to be mixed. After that, purified water was added to the mixture to be 100 Ml total, and then filled in a brown bottle in which it was sterilized, thereby preparing liquid drug.

### INDUSTRIAL APPLICABILITY

Kaempferol and quercetin, which are flavonoids, increase an expression of a HAS gene existing in a skin cell line of human epidermis, thereby promoting a production of hyaluronic acid. Accordingly, the composition for promoting production of hyaluronic acid containing at least one of kaempferol and quercetin according to the invention can be usefully used as a cosmetic composition for increasing skin elasticity and preventing skin dryness or skin aging, or a pharmaceutical composition for treating or preventing a degenerative arthritis.

1. A method for promoting production of hyaluronic acid in an animal subject in need thereof comprising administering an effective amount of kaempferol, quercetin, or the combination thereof to the animal subject.

2. The method according to claim 1, wherein the kaempferol, quercetin, or the combination thereof increases an expression of hyaluronic acid synthase (HAS) gene to promote a production of hyaluronic acid.

3. The method of claim 1, which is for increasing skin elasticity.

4. The method of claim 1, which is for preventing skin dryness.

5. The method of claim 1, which is for preventing skin aging.

6. The method of claim 1, which is for treating or preventing a degenerative arthritis.


SEQUENCE LISTING (6 sequences)

SEQ ID NO: 1 - DNA, 20 bp, Artificial
  Synthetic oligonucleotide
aggtcatgtacacagccttc

SEQ ID NO: 2 - DNA, 20 bp, Artificial
  Synthetic oligonucleotide
cagcagagggacgtagttag

SEQ ID NO: 3 - DNA, 20 bp, Artificial
  Synthetic oligonucleotide
gctaccagtttatccaaacg

SEQ ID NO: 4 - DNA, 20 bp, Artificial
  Synthetic oligonucleotide
ggagtttctgtacattccca

SEQ ID NO: 5 - DNA, 20 bp, Artificial
  Synthetic oligonucleotide
gaggactggtaccatcagaa

SEQ ID NO: 6 - DNA, 20 bp, Artificial
  Synthetic oligonucleotide
accgttctttgcattttaga